(19) United States
(12) Patent Application Publication
(10) Pub. No.: US 2008/0045918 A1
(43) Pub. Date: Feb. 21, 2008

(54) Absorbent article with flat side seams

(75) Inventor: Stacy Driskell, Royersford, PA (US)

Correspondence Address: GOSZ AND PARTNERS LLP, ONE STATE STREET, BOSTON, MA, 02109, US

(73) Assignee: Tyco Healthcare Retail Services, AG

(21) Appl. No.: 11/507,321

(22) Filed: Aug. 21, 2006

(51) Int. Cl. A61F 13/15

(52) U.S. Cl. 604/389; 604/396

(57) ABSTRACT

The present invention discloses a pant-type absorbent article with flat side seams, and a method for manufacturing the same. The absorbent article comprises of a front waist panel, a back waist panel, a crotch region having an absorbent pad and a pair of leg openings disposed between the front panel and the back panel on both sides of the crotch region. The body facing side of one waist panel is bonded to the non-body facing side of the other panel. This kind of bonding ensures that the side seams of the absorbent article stay flat during wear. The bonding is done by using adhesives, hook and loops, or by mechanical/ultrasonic means. In one embodiment of the present invention, the panels are bonded by using a perforated layer of adhesive, thereby enabling easy removal of the article by the wearer.

### BACKGROUND OF THE INVENTION

The present invention relates to the field of absorbent articles and particularly to the field of pant-like absorbent articles for containing body exudates. More particularly, the present invention discloses a pant-like absorbent article with flat side seams.

Absorbent articles have long been used in day-to-day life by different types of users. These articles are available in many forms, such as diapers for babies, adult incontinence articles, feminine hygiene products, and the like. All of these different absorbent articles serve a common purpose of containing various kinds of body exudates.

A typical absorbent article has a top sheet that contacts the body of the wearer, a back sheet that forms the outer portion of the article, and an absorbent core disposed between these two sheets. The top sheet is made from a fluid permeable material that is soft to feel and non-irritable to the skin so as not to cause any discomfort to the wearer. The back sheet is made from a fluid impermeable material so as to block any excess unabsorbed body release from leaking to the outside of the article. The absorbent core is typically made of paper fluff combined with an absorbent material such as super absorbent polymer (SAP), and the like, to absorb body exudates. In addition to this, an acquisition layer may also be present between the top sheet and the core. This layer prevents localized pooling of fluids by spreading the fluid across the entire area of the top sheet.

In particular, pant-like absorbent articles are defined by a back waist panel, a front waist panel and a crotch region. All of these panels are made up of a fluid permeable top sheet and a fluid impermeable back sheet. An absorbent core, in the form of a pad, is sandwiched between these two sheets in the crotch region. The crotch region also has a pair of leg openings disposed between the front and the back waist panel. The back and the front waist panels can be releasably bonded to each other to form side seams.

U.S. Pat. No. 6,113,717, to Vogt et al., discloses a method of making a refastenable, pant-like, disposable absorbent article by attaching a pair of opposed side panels to the side edges of an absorbing chassis such that they extend laterally outward from and between the front and back waist regions of the article.

U.S. Pat. No. 6,667,085, to McNichols, discloses a method of making an absorbent article with prefastened side panels. This is achieved by providing a continuous web of interconnected absorbent articles and joining a pair of opposed side panels to each of the waist regions.

In the aforementioned patents, care is taken to make the absorbent article comfortable for the wearer. The top sheet which comes in direct contact with the body of the wearer is made from a soft and non irritable material. The absorbent core is made from a material with high absorption capacity so that even a thin layer of the core can absorb large amounts of body exudates. This makes the absorbent article light and comfortable to wear. However, protruding side seams still remain a source of discomfort. In the aforementioned patents, side seams are formed by joining a body facing side of one panel with the body facing side of the other panel. As a result, the joined side portions extend laterally outward beyond the side edges of the absorbent article. This causes discomfort to the wearer.

Thus, there is a need for a pant-like absorbent article in which the front and back waist panels are bonded in a fashion such that the bonded portion lays flat during wear.

It is accordingly an objective of the present invention to provide a pant-like absorbent article which does not cause discomfort to the wearer due to protruding side seams.

### SUMMARY OF THE INVENTION

To achieve the aforementioned objective, the present invention discloses an absorbent article with flat side seams and a method of manufacturing the same. In accordance with an embodiment of the present invention, the absorbent article comprises a front waist panel, a back waist panel and a crotch region extending between and connecting the front waist panel and the back waist panel. The crotch region contains an absorbent pad. A pair of leg openings is disposed between the front waist panel and the back waist panel on both sides of the crotch region. The front waist panel and the back waist panel are releasably bonded together such that the body facing side of one panel is bonded to the non body facing side of the other panel. Such bonding ensures that the side seams of the absorbent article stay flat during wear.

The method for manufacturing the absorbent article comprises flipping the front waist panel of the absorbent article up to the back waist panel, folding the side edges of the back waist panel up and over the side edges of the front waist panel, and releasably bonding the side edges of the back waist panel to the side edges of the front waist panel such that the body facing side of the back waist panel is bonded to the non body facing side of the front waist panel.

### BRIEF DESCRIPTION OF THE DRAWINGS

FIG. 1A shows an arrangement of a back sheet web, absorbent pads disposed on the back sheet web, and a top sheet web laid on top of the absorbent pads to form a multi-layered sheet.

FIG. 1B shows a cross sectional view of an individual absorbent article of the invention.

FIG. 2 shows leg holes cut out on the multi-layered sheet of the invention.

FIG. 3 shows individual absorbent articles after the absorbent articles are placed spaced apart.

FIG. 4 shows the arrangement of individual articles such that the short waist panel is the leading edge.

FIG. 5 shows the flipping of the short waist panel of the absorbent article onto the long waist panel.

FIG. 6 shows the folding of the long waist panel edges over the short waist panel edges.

FIG. 7 shows an embodiment of the present invention wherein a perforated adhesive is applied on the side edges of the waist panel.

FIG. 8 is a method flowchart outlining the steps involved in manufacturing the absorbent article in accordance with an embodiment of the invention.

### DETAILED DESCRIPTION

The present invention discloses a pant type absorbent article with flat side seams and a method for manufacturing the same. The front and back waist panels of the absorbent article are bonded in such a way that the body-facing side of one panel faces the non-body facing side of the other panel. Such bonding ensures that the side seams of the absorbent article stay flat during wear.

In the following specification, specific embodiments of the present invention have been described. However, one of ordinary skill in the art will appreciate that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as critical, required, or essential features or elements of any or all the claims.

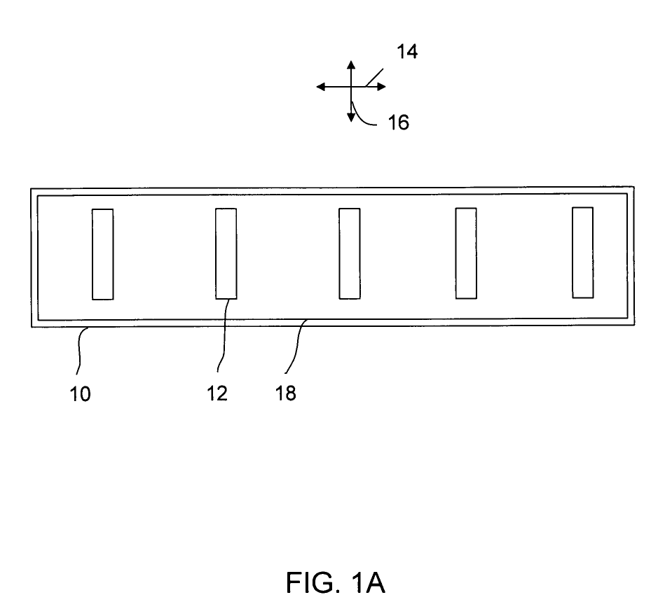

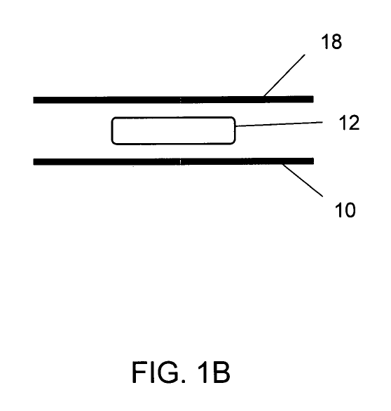

FIG. 1A shows a rectangular back sheet web 10 made from a fluid impervious material such as polyethylene or polypropylene. Back sheet web 10 is cut into multiple individual absorbing articles. Absorbent pads 12 made up of one or more layers of absorbent materials are attached to back sheet web 10 at regular spacing. The attachment is done in a manner such that the length of absorbent pads is parallel to machine direction (MD) axis 14, and perpendicular to cross directional (CD) axis 16 of the back sheet. The distance between two adjacent absorbent pads 12 depends on the required size of the individual absorbent article.

A top sheet web 18 made from a fluid permeable material is then attached on top of absorbent pads 12 laid on back sheet web 10. Top sheet web 18 is made from a soft and non irritable material to avoid any discomfort to the wearer. Therefore, in this arrangement, the absorbent pad is sandwiched between back sheet web 10 and top sheet web 18 to form a multilayered sheet. This multilayered sheet is cut into multiple individual absorbent articles; each individual article having a top sheet, a back sheet and an absorbent pad interposed between the top sheet and the back sheet. It will be apparent to one skilled in the art that the multilayered sheet may have additional layers disposed between the top sheet web and the back sheet web. For example, in one embodiment, the multilayered sheet has a porous acquisition layer incorporated between the top sheet web and the absorbent pads. The acquisition layer in the form of a fibrous wadding, a carded fibrous web or other type of fibrous material, can temporarily store fluids before they are finally absorbed by the absorbent pads.

FIG. 1B shows a cross sectional view of an individual absorbent article made by cutting the multilayered sheet into discrete articles. Absorbent pad 12 is shown to be sandwiched between the fluid permeable top sheet and the fluid impervious back sheet.

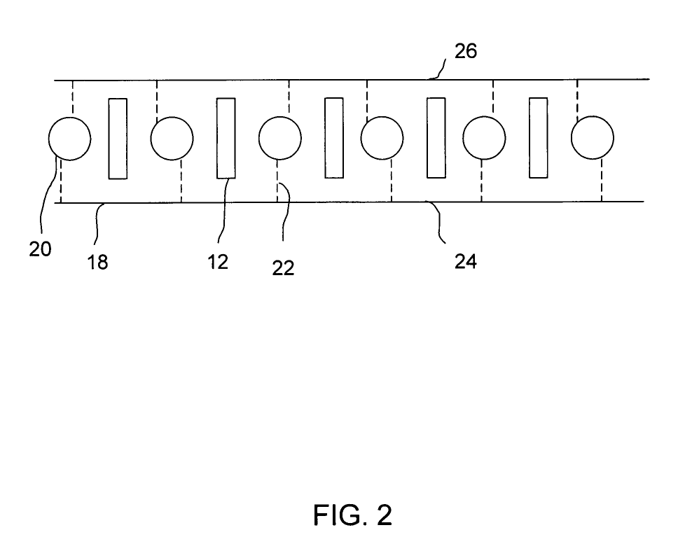

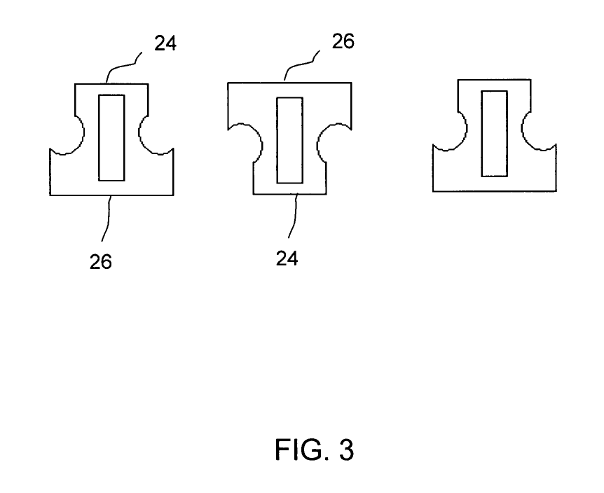

Circular leg holes 20 are cut out from the multilayered sheet in between the absorbent pads as shown in FIG. 2. The size of the leg hole cut depends on the size of leg hole required in the article. Depending upon the requirements, leg holes can be cut in either oval or elliptical shapes as well. In order to cut the multi-layered sheet into individual articles, cuts are made along the dotted lines 22 as shown in FIG. 3. Each cut starts from an edge of the multilayered sheet and ends at a leg hole, and is parallel to the MD axis. The cuts are made so as to form multiple individual absorbent articles. The edges of the multilayered sheet form the waist panels of the individual absorbent article, while the absorbent pads form the crotch region when the article is worn. The multilayered sheet is cut in such a way that one waist panel 24 of the resulting article is shorter than the other panel 26. When the absorbent article is worn, the short waist panel may form the front waist panel, while the long waist panel may form the back waist panel.

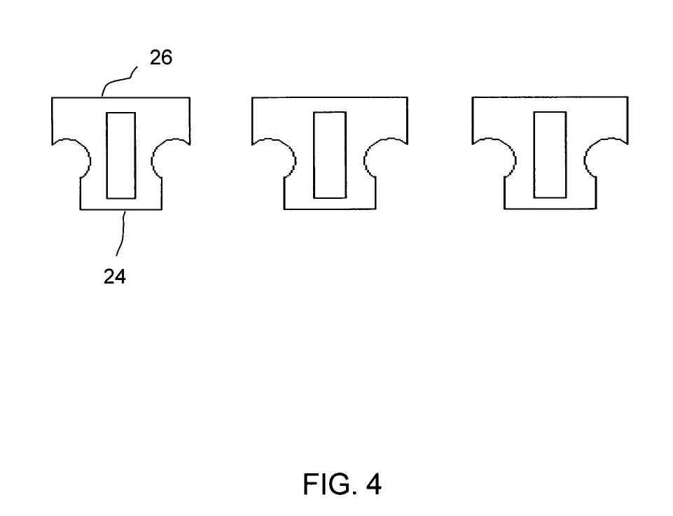

The individual absorbent articles are then separated as shown in FIG. 3. When the individual articles are separated and placed apart, short waist panel 24 and long waist panel 26 form the opposite edges of the absorbent article. The individual articles are then arranged in a manner such that short waist panel 24 forms the leading edge while long waist panel 26 forms the trailing edge as shown in FIG. 4.

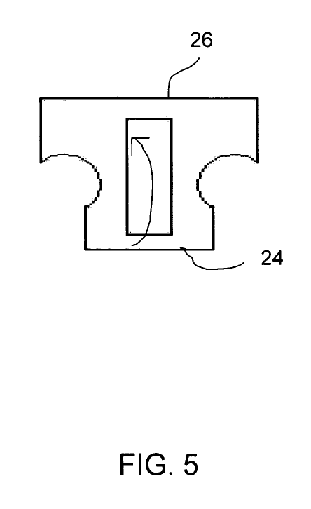

FIG. 5 shows the flipping of short waist panel 24 onto long waist panel 26.

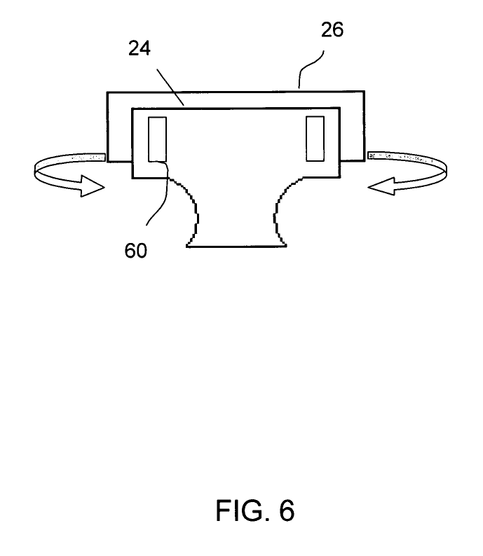

FIG. 6 shows the folding of long waist panel edges over short waist panel edges. The edges are releasably bonded with the help of an adhesive 60 applied on one of the waist panels. In FIG. 6, adhesive 60 is applied on the side edges of the short waist panel. It will be apparent to one skilled in the art that the bonding may also be done using mechanical and/or ultrasonic means, or by a hook and loop arrangement. Further, the bonds may be point bonds, dashed lines, continuous lines, discontinuous lines and combinations thereof.

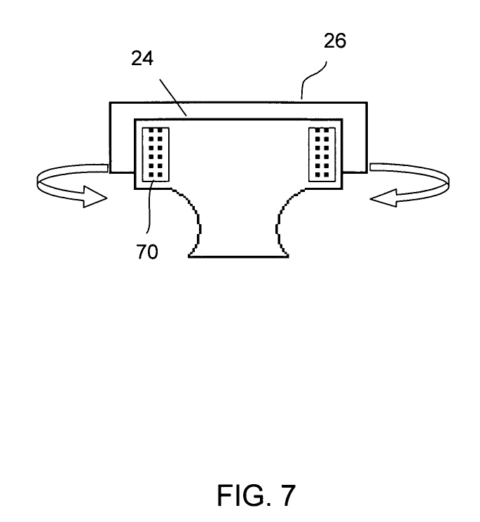

FIG. 7 shows an absorbent article in accordance with an alternate embodiment of the present invention. In this embodiment, the adhesive applied on the side edge of the waist panel is perforated 70. This makes the side edges ‘tear away’ and enables easy removal of the absorbent article from the body of the wearer.

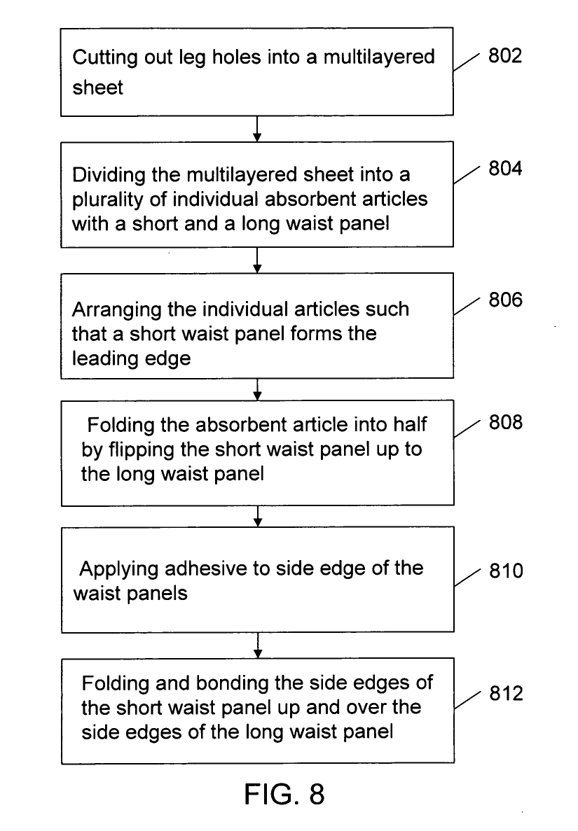

FIG. 8 is a method flowchart outlining the steps involved in manufacturing the absorbent article in accordance with an embodiment of the invention. The absorbent articles are cut from a multilayered sheet comprising a fluid impervious back sheet web, a fluid pervious top sheet web, and a plurality of absorbent pads disposed between the back sheet web and the top sheet web. The number of absorbent pads disposed between the back sheet web and the top sheet web corresponds to the number of absorbent articles that can be formed from the multilayered sheet.

At step 802, leg holes are cut out into the multilayered sheet. The leg holes are cut such that one leg hole is positioned between two adjacent absorbent pads. At step 804 the multilayered sheet is divided into multiple individual absorbent articles. This division is made by cutting the multilayered sheet such that a cut extends from a length of the rectangular multilayered sheet to the circumference of the leg hole. The lengths of the multilayered sheet form the waist panels of the individual absorbent article, while the absorbent pads form the crotch region when the article is worn. The multilayered sheet is cut in such a way that one waist panel of the resulting article is shorter than the other. At step 806 the individual absorbent articles are separated and arranged such that the short waist panel forms the leading edge while the long waist panel forms the trailing edge. At step 808 the absorbent article is folded in half by flipping the short waist panel up to the long waist panel. At step 810 a bonding material such as an adhesive is applied to the side edges of the short waist panel. At step 812, the side edges of the long waist panel are folded up and over the side edges of the short waist panel. An adhesive is used for releasably bonding the side edges of the short waist panel with the side edges of the long waist panel.

It will be apparent to one skilled in the art that different bonding arrangements may be used for bonding of the waist panels. In different embodiments, adhesives, hook and loop arrangements, mechanical bonds and ultrasonic bonds may be used for such bonding.

The aforementioned embodiments and examples are meant to be solely for explanatory purposes, and are in not intended to limit the scope of the invention. Various other configurations are possible within the spirit of the invention. The invention is intended to cover all such embodiments within the boundaries defined by the appended claims.

What is claimed is:

1. An absorbent article comprising:
a front waist panel;
a back waist panel;
a crotch region having an absorbent pad, the crotch region extending between and connecting the front waist panel and the back waist panel; and
a pair of leg openings disposed between the front waist panel and the back waist panel;
wherein the front waist panel and the back waist panel are releasably bonded together such that the body facing side of one panel is bonded to the non-body facing side of the other panel.

2. The absorbent article as claimed in claim 1, wherein the body facing side of the front waist panel is bonded to the non-body facing side of the back waist panel.

3. The absorbent article as claimed in claim 1, wherein the body facing side of the back waist panel is bonded to the non-body facing side of the front waist panel.

4. The absorbent article as claimed in claim 1, wherein the front waist panel and the back waist panel are bonded together by at least one of an adhesive, a mechanical bond, and an ultrasonic bond.

5. The absorbent article as claimed in claim 1, wherein the front waist panel and the back waist panel are bonded together by a perforated adhesive.

6. The absorbent article as claimed in claim 1, wherein the front waist panel and the back waist panel are bonded together by a hook and loop arrangement.

7. A method for manufacturing an absorbent article, the absorbent article being cut from a multilayered sheet comprising a fluid impervious back sheet web, a fluid pervious top sheet web, and a plurality of absorbent pads disposed between the back sheet web and the top sheet web, the method comprising
cutting out leg holes into the multilayered sheet, the leg holes being placed between two adjacent absorbent pads;
dividing the multilayered sheet into a plurality of individual absorbent articles wherein the resulting individual articles each have a short waist panel, a long waist panel, a crotch region having an absorbent pad, and a pair of leg openings disposed between said short panel and said long panel;
separating and arranging the individual absorbent articles such that short waist panel forms a leading edge;
flipping the short waist panel up to the long waist panel;
applying bonding material on side edges of at least one of the short and long waist panels; and
folding and bonding the side edges of the long waist panel up and over the side edges of the short waist panel.

8. The method as claimed in claim 7, wherein a body facing side of the long waist panel is bonded to a non-body facing side of the short waist panel.

9. The method as claimed in claim 7, wherein a body facing side of the short waist panel is bonded to a non-body facing side of the long waist panel.

10. The method as claimed in claim 7, wherein dividing the multilayered sheet comprises cutting the multilayered sheet such that a cut extends from a length of the multilayered sheet to a circumference of the leg hole.

11. A method for manufacturing an absorbent article having a front waist panel, a back waist panel, a crotch region extending between and connecting the front waist panel and the back waist panel, the crotch region containing an absorbent pad, and a pair of leg openings disposed between the front waist panel and the back waist panel, the method comprising:
flipping the front waist panel up to the back waist panel;
folding side edges of the back waist panel up and over the side edges of the front waist panel; and
releasably bonding the side edges of the back waist panel to the side edges of the front waist panel, wherein the body facing side of the back panel is bonded to the non-body facing side of the front panel.
